(19) United States
(12) Patent Application Publication
(10) Pub. No.: US 2023/0248985 A1
(43) Pub. Date: Aug. 10, 2023

(54) SYSTEMS AND METHODS FOR DIAGNOSING AND TREATING FIBRILLATION

(71) Applicant: Arrhythmia Dynamics LLC, Madison, CT (US)

(72) Inventor: James P. Hummel, Madison, CT (US)

(21) Appl. No.: 18/013,125

(22) PCT Filed: Jun. 30, 2021
(86) PCT No.: PCT/US2021/039988

§ 371 Date: Dec. 27, 2022

Related U.S. Application Data
(60) Provisional application No. 63/046,404, filed on Jun. 30, 2020.

(51) Int. Cl. A61N 1/39; A61B 5/361; A61N 1/362; A61B 5/00

(52) U.S. Cl. CPC A61N 1/39622; A61B 5/361; A61N 1/395; A61N 1/3621; A61B 5/686

(57) ABSTRACT

Methods and systems for detecting stability of the arrhythmia or fibrillation, determining whether defibrillation or pacing is needed to disrupt the fibrillation, and, if so, optimizing the timing of low energy therapies to improve efficacy of low energy therapy for defibrillation. By transforming electrogram signals into a discrete series of electrogram conformations, recurrence variables of the electrogram signal can be determined that are highly indicative of sources of the arrhythmia, that predict the likelihood of spontaneous termination of the arrhythmia, and that detect the optimal timing of low energy treatment to terminate the arrhythmia.

### RELATED APPLICATION

This present application claims the benefit of U.S. Provisional Application No. 63/046,404 filed Jun. 30, 2020, which is hereby incorporated in its entirety by reference.

### TECHNICAL FIELD

Embodiments of the present disclosure relate generally to cardiology, and more specifically to the detection, treatment, and ultimately termination of fibrillations.

### BACKGROUND

Abnormal or irregular cardiac rhythms are called cardiac arrhythmias. These disturbances disrupt the heart's electrical signals and can cause the heart to beat too fast, too slowly, or in an abnormal way. Cardiac arrhythmias are associated with abnormal initiation of a wave of cardiac excitation, abnormal propagation of a wave of cardiac excitation, or some combination of the two. Cardiac arrhythmias can manifest themselves in many different ways, making it difficult to determine the mechanism of an arrhythmia.

Arrhythmias can be classified as reentrant versus non-reentrant arrhythmias. In reentry, cardiac tissue is repetitively excited by a propagating wave circulating around an obstacle, known as anatomical reentry, or circulating freely in the tissue as a spiral or scroll wave, functional reentry.

Tachycardia is a reentrant arrhythmia in which a subject's heart rate is over 100 beats per minute. There are many different types of tachycardia grouped by the part of the heart responsible for the abnormality, such as, for example, atrial fibrillation, atrial flutter, supraventricular tachycardia, ventricular tachycardia, and ventricular fibrillation.

Specifically, ventricular fibrillation, or VF, is considered a very serious cardiac arrhytmia, and a life threatening medical event. During VF, disordered electrical activity causes the heart's lower chambers or ventricles to quiver, or fibrillate, instead of contracting (or beating) normally. This prohibits the heart from pumping blood, causing collapse and cardiac arrest. VF can be treated with arrhythmia medications, which can help control rhythm disturbances, and/or with an implantable cardioverter defibrillator (ICD), which can correct abnormal heart rhythms via a single high energy shock. Arrhythmia medications can include, for example, antiarrhythmic drugs, calcium channel blockers, beta blockers, and anticoagulants.

An ICD is a battery-powered device placed under the skin that keeps track of a subject's heart rate via thin wires that connect the ICD to the heart. If an abnormally fast or chaotic heart rhythm is detected, the device will deliver an electric shock to restore a normal heartbeat. Certain ICDs can also have the dual functionality of a pacemaker in which it sends small electrical signals to the heart when the detected heartbeat is too slow.

Recently, there is a strong association between ICD shocks and increased mortality. Some of this is undoubtedly due to ICD shocks serving as a marker for more advanced disease. However, any of the following can also contribute to an increased mortality: inappropriate shocks, i.e. the shocks are too large for the abnormal heart rhythm; myocardial damage with increased biomarkers of injury due to shocks; myocardial stunning and cell death via electroporation from shocks; and/or psychological distress from pain associated with shocks. In this respect, ICD programming has been found to aid in the reduction of inappropriate shocks to reduce mortality.

Besides high energy single shock therapy, other strategies to terminate ongoing reentrant tachycardias and fibrillation are being explored and can include, for example, anti-tachycardia pacing (ATP), multistage electrotherapy, a series of low energy biphasic and multiphasic shocks followed by ATP after organization, low energy anti-fibrillation pacing (LEAP), and multisite photostimulation.

Atrial fibrillation, or AF, is a dynamic, nonstationary system with multiple potential mechanisms including spiral wave reentry and multiple wavelet reentry. AF is the most common type of tachyarrhythmia encountered in clinical practice. Besides antiarrythmic medication, catheter ablation is currently a standard therapy in patients. Specifically, pulmonary vein isolation (PVI) is a mainstream catheter ablation technique for paroxysmal AF. For patients who have not responded to PVI, substrate modification with complex fractionated electrogram (CFE) ablation may be necessary to treat persistent AF.

It has been demonstrated that dynamics of local wavefront direction (WD) may give important information and insight into local dynamics and the underlying mechanism of fibrillation. Specifically, bipolar electrogram morphology yields important information about the direction of wavefront propagation relative to the electrodes. Differentiation of areas exhibiting highly periodic or intermittently periodic behavior from regions of chaos could be important in identifying sources of either VF or AF.

Recurrence quantification analysis (RQA) is a nonlinear tool that readily differentiates periodicity from chaos and quantifies stability, determinism and other features of dynamical systems, and thus could be helpful to differentiate periodic behavior from chaotic behavior. For example, details of RQA for use to differentiate regions of spiral wave reentry from wavelet breakup during atrial fibrillation was recently described in a publication entitled “A method for quantifying recurrent patterns of local wavefront direction during atrial fibrillation,” to James P. Hummel et al. (Computers in Biology and Medicine 89 (2017) 497-504), attached hereto as Appendix A, and “New skip parameter to facilitate recurrence quantification of signals comprised of multiple components,” to James P. Hummel et al. (Chaos 28, 085718 (2018): https://doi.org/10.1063/1.5024845), attached hereto as Appendix B, and “Recurrence quantification analysis of complex-fractionated electrograms differentiates active and passive sites during atrial fibrillation,” to Baher et al. (J. Cardiovasc Electrophysiol. 2019; 30:2229-2238) attached hereto as Appendix C, all of which are incorporated herein by reference in their entireties. To date, these methods have been utilized to optimize individualization of ablation strategies in AF patients.

There remains a need to use such strategies to develop algorithms and devices that better predict the dynamics of arrhythmia to optimize therapy strategy and timing of the selected therapy.

### SUMMARY

Embodiments of the present disclosure are directed to methods and systems for detecting stability of the arrhythmia or fibrillation, determining whether defibrillation or pacing is needed to disrupt the fibrillation, and, if so, optimizing the timing of low energy therapies to improve efficacy of low energy therapy for defibrillation. By transforming electrogram signals into a discrete series of electrogram conformations, recurrence variables of the electrogram signal can be determined that are highly indicative of sources of the arrhythmia, that predict the likelihood of spontaneous termination of the arrhythmia, and that detect the optimal timing of low energy treatment to terminate the arrhythmia.

More specifically, wavefront propagation throughout a dielectric medium can be described as a system of differential equations of multiple variables yielding nonlinear dynamics and chaotic solutions under certain conditions. Under normal clinical circumstances we do not have knowledge of the state of many variables over time. However, information about the dynamics of the system may still be ascertained from knowledge of a single or limited number of variables, by placing a discrete number of sensors within one or more chambers of the heart, and embedding the time series of these variable(s) into a multidimensional phase space. This Poincaré plot can in turn be used generate recurrence plots. The recurrence plots (or cross-recurrence plots between several variables) are then used to detect varying extents of organization with periods of high organization, in which there is low wavebreak, a low number phase singularities, and high coupling between different regions of the myocardial tissue, and low organization. The dynamics of the system and extent of organization will also be determined using techniques including but not limited to regional phase mapping, via a plurality of discrete sensing electrodes placed within one or more chambers of the heart, with measurements of synchronization and coherence between regions. In embodiments, analysis of phase synchronization is used to detect period phase synchronization, intermittent phase synchronization, or repeated episodes of phase synchronization that occur over time, which may predict its probability of spontaneous termination of the fibrillation without the need for low or high energy shock. For the sake of efficiency, the term “periodic” herein means phase synchronizations repeating at regular intervals, phase synchronizations repeating at irregular intervals, and intermittent phase synchronizations.

In embodiments, detection of phase synchronizations may also allow optimization of timing of delivery of low energy therapies or other therapies that otherwise would fail if the system was unable to synchronize sufficient myocardium in heart chambers of interest. In this embodiment, if therapy is timed to a period when significant synchronization is already present, it may have a higher probability of achieving defibrillation, i.e. higher efficacy. In other embodiments, if minimal or no phase synchronization is detected, higher energy shock may be necessary to achieve defibrillation.

The systems and methods can be used, for instance, to diagnose and classify sources of arrhythmia or fibrillation in the subject, to detect phase synchronizations to determine and to predict the optimal method of and timing for achieving defibrillation in the subject, e.g. through spontaneous termination (no energy), low energy therapy such as pacing or low energy shock, or high energy shock.

The systems and methods of the disclosure can be used in combination with atrial defibrillators known to one of ordinary skill in the art to detect a-fib episodes with a high probability of self-termination in which therapy is withheld, to detect episodes of a-fib which are similar to flutter and where anti-tachycardia pacing (ATP) is predicted to be highly effective, and to detect episodes of a-fib in which low energy shocks are necessary and optimize timing of low energy shocks, thereby improving the efficacy of the low energy therapy. In some embodiments, the determination to withhold therapy can be in a range of a few seconds to a day or more, depending on a severity of symptoms in a particular subject and/or the probability of self-termination.

In other embodiments, the systems and methods of the disclosure can be used in combination with ICDs known to one of ordinary skill in the art to detect VF or ventricular tachycardia episodes in which it is determined to withhold therapy for a short time (e.g. 8-10 seconds or less) if the episode is very unstable to determine whether it will self-terminate, to identify which episodes are likely to respond with ATP or other therapies instead of high energy shock, and to identify which episodes are likely to respond to low energy therapy and optimize the timing of such therapy.

According to embodiments, a defibrillation optimizing control system for treating a heart rhythm disorder in a subject generally comprises a sensor operably coupled to the subject and configured to collect data from the subject, and a therapy control sub-system configured to receive data from the sensor, wherein the data comprises heart activity signals of the subject. The therapy control sub-system is configured to analyze the heart activity signals to detect an arrhythmia, and when an arrhythmia is present, the therapy control sub-system is configured to detect periodic phase synchronizations to determine a probability of the arrhythmia self-terminating within a period of time, and based on the probability of self-terminating, to determine a defibrillation therapy. The therapy control sub-system is also configured to transmit information to effect a defibrillation therapy within the subject or withhold a defibrillation therapy for a period of time. In embodiments, the therapy control sub-system is configured to transmit information to withhold the defibrillation therapy when the probability is above a first threshold. In an embodiment, the heart rhythm disorder is atrial fibrillation and the first threshold is in range from about 10% to about 90%, and the period of time is in a range of about 10 seconds to 240 minutes. In another embodiment, the heart rhythm disorder is ventricular fibrillation and the first threshold is about 90% or greater, and the period of time is in a range of about 1 to about 10 seconds.

In embodiments, when the probability is at or below the first threshold, the sub-system is configured to determine an extent of organization, and to transmit information to effect delivery of a first therapy or to effect delivery of a second therapy depending on the extent of organization. The first therapy comprises energy delivery at a first energy level, and the second therapy comprises delivery of a second therapy at a second energy level greater than the first energy level. In embodiments, when the first therapy is selected, the system is configured to optimize a delivery time of the first therapy at the first energy level. The delivery time can be periods of tissue synchronization.

In alternative embodiments, the first therapy comprises anti-tachycardia pacing by a series of electrical impulses delivered to a heart muscle of the subject to restore a normal heart rate and heart rhythm for the subject. In another embodiment, the first therapy comprises multistage electrotherapy by a series of biphasic and/or multiphasic shocks, followed by anti-tachycardia pacing by a series of electrical impulses delivered to a heart muscle of the subject to restore a normal heart rate and heart rhythm for the subject, In another embodiment, the low energy anti-fibrillation pacing, and in yet another embodiment, the first therapy comprises multisite photostimulation.

In embodiments, the heart activity signals comprise intracardiac electrogram signals, and the sub-system is configured to analyze the signals using real-time recurrence quantitative analysis. The signals are analyzed to determine an extent of signal coupling present between different regions of heart tissue over time. The extent of signal coupling and/or phase synchronization is measured using one or more tools selected from the group consisting of phase mapping, cross-recurrence, recurrence networks, synchronization, coherence, and combinations thereof, and/or the extent of signal coupling is measured using parameters obtained from recurrence networks, multivariate recurrence plots or joint recurrence plots including synchronization measures derived from these plots including correlation of probability of recurrence and joint probability of recurrence. In other embodiments, the extent of signal coupling is measured using one or more tools selected from the group consisting of synchronization measures including Kuramoto order parameter, interdependence index, network transitivity, and cross transitivity. The variables measured by the one or more tools are selected from the group consisting of determinism, % recurrence, entropy, distribution and trends of diagonal and vertical line lengths, trapping time, Lyapunov exponent, coherence, phase matching, and combinations thereof.

In embodiments, the sub-system comprises circuitry within an implantable device, wherein the implantable device is configured to deliver the defibrillation therapy. In other embodiments, the sub-system comprises an external device wirelessly coupleable to the sensor. In embodiments, the sensor is configured to collect data from the subject and transmit information to effect the defibrillation therapy within the subject, while in other embodiment, an effector separate from the sensor, the effector being configured to effect the defibrillation therapy within the subject. In embodiments, the sensor comprises at least two electrodes, and the effector comprises at least two electrodes. In embodiments, the at least two electrodes of the sensor, the effector, or both comprises leads. In embodiments, the sensor and the subsystem comprise a single implantable device, while in other embodiments, the sensor and the subsystem comprise two separate devices, and at least one of the two separate devices is implantable within the subject. In some embodiments, the sensor, the effector, and the subsystem comprise a single implantable device, while in other embodiments, the sensor, the effector, and the subsystem comprise at least two separate devices. At least one of the two separate devices is implantable within the subject.

According to embodiments, a method for optimizing defibrillation related to a heart rhythm episode in a subject can comprise receiving, from a sensor operably coupled to the subject, sensor data related to a heart rhythm episode; evaluating, via an optimization engine, the sensor data for periodic phase synchronizations; determining, based on the evaluation step, a probability of the arrhythmia self-terminating within a period of time; selecting, based on the probability of self-terminating, to withhold a defibrillation therapy for a period of time if the probability is above a first threshold, to deliver a first defibrillation therapy, or to deliver a second defibrillation therapy if the probability is at or below the first threshold; and delivering the first defibrillation therapy or the second defibrillation therapy when withholding a defibrillation therapy is not selected. In embodiments, the sensor data comprises intracardiac electrogram signals.

In embodiments, the first defibrillation therapy comprises delivery of energy at a first energy level and the second defibrillation therapy comprises delivery of energy at a second energy level higher than the first energy level, and the method includes optimizing a delivery time of the first energy level when the first defibrillation therapy is selected. The delivery time can be at periods of tissue synchronization.

In embodiments, the first defibrillation therapy comprises anti-tachycardia pacing by a series of electrical impulses delivered to a heart muscle of the subject to restore a normal heart rate and heart rhythm for the subject. In other embodiments, the first defibrillation therapy comprises multistage electrotherapy by a series of biphasic and/or multiphasic shocks, followed by anti-tachycardia pacing by a series of electrical impulses delivered to a heart muscle of the subject to restore a normal heart rate and heart rhythm for the subject. In yet other embodiment, the first defibrillation therapy comprises low energy anti-fibrillation pacing or multisite photostimulation. In embodiments, the signals are analyzed to determine an extent of signal coupling present between different regions of heart tissue over time.

In an embodiment, the heart rhythm episode is an atrial fibrillation episode, and the first threshold is a probability of fibrillation self-terminating greater than 10% within a near term window of 1 to 240 minutes. In an embodiment, the probability and near-term window duration is programmable as a function of an input patient symptom severity score such that a lower patient symptom severity score will result in lower probability threshold and longer near term window. The input patient symptom severity score can be a scale from 1 to 5, wherein 1 denotes minimal symptoms and 5 denotes severe symptoms. In embodiments, when the probability is at or below the first threshold, the selecting step further comprises determining whether the atrial fibrillation resembles a flutter; and delivering energy at a first energy level when the atrial fibrillation resembles a flutter or delivering energy at a second energy level higher than the first energy level when the atrial fibrillation does not resemble a flutter.

In another embodiment, the heart rhythm episode is a ventricular tachycardia or a ventricular fibrillation episode. When the probability is at or below the first threshold, the selecting step further comprises determining a likelihood of termination of the episode by delivery of energy at a first energy level. The therapy will be deferred during ventricular fibrillation if analysis yields high probability of fibrillation self-terminating is in a range of about 80% to about 95% within a short near term window of a range of about 4 to about 15 seconds.

In embodiments, when the probability is above the first threshold, withholding delivery of energy is selected, the method further comprises waiting for the period of time to determine whether the episode self-terminated; analyzing the sensor data to determine an extent of signal coupling present between different regions of heart tissue over time; and selecting, if the episode does not self-terminate within the period of time, depending on the extent of coupling, the first defibrillation therapy or the second defibrillation therapy.

In embodiments, a defibrillation optimizing control system for treating tachycardia in a subject can comprise a sensor, the sensor operably coupled to the subject in various regions of the atria, the ventricles, or both; and a therapy control sub-system configured to receive data from and transmit instructions to the sensor, wherein the data comprises intracardiac electrogram signals; wherein the therapy control sub-system is configured to analyze the signals to detect arrhythmia, and when present, is configured to determine an extent of coupling between the signals, and based on the extent of coupling, determine whether to deliver or withhold a defibrillation therapy comprising energy delivery.

In yet other embodiments, the systems and methods of the disclosure can be used in known deep brain stimulation therapies to regulate other fibrillations of the body, such as in the brain, used for treatment of neurological conditions such as epilepsy and/or movement disorders like Parkinsons.

The above summary is not intended to describe each illustrated embodiment or every implementation of the present invention. The detailed description that follows more particularly exemplifies these embodiments.

### BRIEF DESCRIPTION OF THE DRAWINGS

Subject matter hereof may be more completely understood in consideration of the following detailed description of various embodiments in connection with the accompanying figures, in which:

FIG. 1 is a schematic diagram of a system for optimizing defibrillation, according to an embodiment.

FIG. 2 is a block diagram of a therapy sub-system of FIG. 1, according to an embodiment.

FIG. 3 is a flowchart of a method for optimizing defibrillation related to an atrial fibrillation episode, according to an embodiment.

FIG. 4 is a flowchart of a method for optimizing defibrillation related to ventricle tachycardia or ventricle fibrillation, according to an embodiment.

FIG. 5A is an echocardiogram (ECG).

FIG. 5B is a Poincaré plot generated from the ECG of FIG. 8A.

FIG. 6A depicts phase space trajectories of two coupled Roessler systems;

FIG. 6B is a cross recurrence plot (CRP) of the two systems is displayed in FIG. 6A.

FIGS. 7A-7C depict different trends interpreted from recurrence plots.

FIG. 8 is a flowchart of a method for optimizing treatment related to AF, according to an embodiment.

FIG. 9 is a flowchart of a method for optimizing treatment related to VF, according to an embodiment.

FIGS. 10A-10C are recurrence plots depicting stable coupling (FIG. 10A), stable uncoupling (FIG. 10B), and periodic instabilities (FIG. 10C).

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

As mentioned in the Summary, embodiments of the present disclosure can be used in combination with atrial defibrillators known to one of ordinary skill in the art to detect and treat a-fib episodes and/or can be used in combination with ICDs known to one of ordinary skill in the art to detect v-fib or ventricular tachycardia episodes. The systems and methods can be used to diagnose and classify sources of arrhythmia or fibrillation in a subject, to detect phase synchronizations to determine, and to predict the optimal method of and timing for achieving defibrillation in the subject, e.g. through spontaneous termination (no energy) or application of one or more therapies including low energy therapy such as pacing or low energy shock, and high energy shock. Ultimately, by better predicting the real-time and dynamic behavior of the cardiac arrhythmia, the systems and methods employ alternative therapies or no therapy, thereby reducing the occurrence of high energy shocks, and the risks associated therewith.

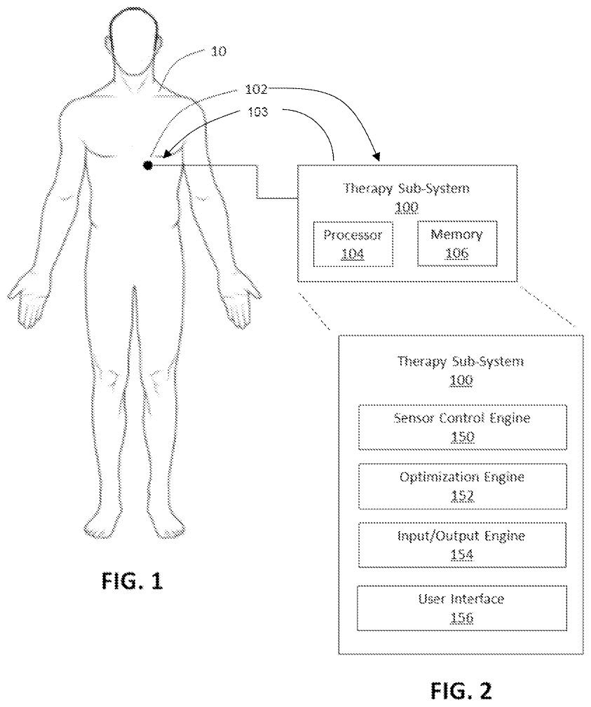

Referring to FIG. 1, a schematic diagram of a system for optimizing defibrillation is depicted, according to an embodiment. The system generally comprises a therapy sub-system 100 and at least one sensor 102, and at least one effector or actuator 103. In one embodiment, sensor 102 is the same as effector 103. In another embodiment sensor 102 is a separate component than effector 103, as will be described in more detail below.

Therapy sub-system 100 is configured to receive and analyze data related to or measured by sensor 102, and in some embodiments, to further optimize defibrillation therapy. In an embodiment, therapy sub-system 100 generally comprises a processor 104 and a memory 106 operably coupled to processor 104.

Processor 104 comprises a programmable device that accepts digital and/or analog data as input, is configured to process the input according to instructions or algorithms, and provides results as outputs. In an embodiment, a processor can be a central processing unit (CPU) configured to carry out the instructions of a computer program. A processor is therefore configured to perform at least basic arithmetical, logical, and input/output operations. In another embodiment, a processor can be an integrated circuit or logic circuit to carry out basic arithmetical and logical operations in real time on the input and provide real time output.

Memory 106 operably coupled to processor 104 can comprise volatile or non-volatile memory as required by the coupled processor to not only provide space to execute the instructions or algorithms, but to provide the space to store the instructions themselves. In embodiments, volatile memory can include random access memory (RAM), dynamic random-access memory (DRAM), or static random-access memory (SRAM), for example. In embodiments, non-volatile memory can include read-only memory, flash memory, ferroelectric RAM, hard disk, floppy disk, magnetic tape, or optical disc storage, for example. The foregoing lists in no way limit the type of memory that can be used, as these embodiments are given only by way of example and are not intended to limit the scope of the invention.

Various embodiments of systems, and the corresponding methods of configuring and operating the system, can be performed in cloud computing, client-server, or other networked environments, or any combination thereof. The components of the system can be located in a singular “cloud” or network, or spread among many clouds or networks. End-user knowledge of the physical location and configuration of components of the system is not required.

Sensor 102 is operably coupled to patient 10. In an embodiment, sensor 102 can comprise one or more electrodes, leads, or other sensing element. Sensor 102 can make up part of an implantable device, such as in an implantable cardioverter-defibrillator (ICD) or atrial defibrillator. For example, an electrode or lead can be attached or otherwise operably coupled to the right ventricle (RV). In another example, leads or electrodes are attached or otherwise operably coupled to the right atrium (RA) and the RV. In another example, two or three electrodes or leads are positioned in the RA, the RV, and the left ventricle (LV). Accordingly, the electrodes or leads can be electrically coupled to a pulse generator configured to selectively apply energy therapies. Alternatively, energy therapies can be applied via a separate set of electrodes or leads of an effector, as will be described in more detail, infra. Further, such electrodes or leads can also be utilized to sense areas in and around the tissue in which they are positioned.

In an embodiment, sensor 102 can comprise additional sensing hardware such as an optical, thermal, or wavelength sensor. In another example, sensor 102 can include a transducer or an electrode configured to measure intracardiac atrial fibrillation (electrogram) signals from patient 10. Such sensors are given by way of example only and are not intended to be limiting.

Referring to FIG. 2, a block diagram of therapy sub-system 100 of FIG. 1 is further depicted, according to an embodiment. For ease of illustration, processor 104 and memory 106 are not re-copied in FIG. 2, but one of skill in the art will readily appreciate that the engines of therapy sub-system 100 can be implemented using processor 104 and memory 106. In an embodiment, therapy sub-system 100 generally comprises a sensor control engine 150, an optimization engine 152, an input/output engine 154, and a user interface 156.

The engines described herein can be constructed, programmed, configured, or otherwise adapted, to autonomously carry out a function or set of functions. The term engine as used throughout this document is defined as a real-world device, component, or arrangement of components implemented using hardware, such as by an application specific integrated circuit (ASIC) or field-programmable gate array (FPGA), for example, or as a combination of hardware and software, such as by a microprocessor system and a set of program instructions that cause the engine to implement the particular functionality, which (while being executed) transform the microprocessor system into a special-purpose device. An engine can also be implemented as a combination of the two, with certain functions facilitated by hardware alone, and other functions facilitated by a combination of hardware and software. Accordingly, each engine can be realized in a variety of physically embodied configurations, and should generally not be limited to any particular implementation exemplified herein, unless such limitations are expressly called out. In addition, an engine can itself be composed of more than one sub-engines, each of which can be regarded as an engine in its own right. Moreover, in the embodiments described herein, each of the various engines corresponds to a defined autonomous functionality; however, it should be understood that in other contemplated embodiments, each functionality can be distributed to more than one engine. Likewise, in other contemplated embodiments, multiple defined functionalities may be implemented by a single engine that performs those multiple functions, possibly in parallel or series with, and/or complementary to other functions, or distributed differently among a set of engines than specifically illustrated in the examples herein.

Sensor control engine 150 is configured to communicate with sensor 102. In an embodiment, sensor control engine 150 is communicatively coupled to sensor 102 to receive data from sensor 102, as well as transmit data (e.g. therapy commands) to sensor 102.

Optimization engine 152 is configured to receive data from sensor control engine 150, determine appropriate an appropriate therapy for patient 10, and communicate the appropriate therapy to effector 103. In embodiments, optimization engine 152 can determine the various periods of organization of fibrillation, including periods of high organization (low wavebreak, low number phase singularities, high coupling between different regions) and low organization. In embodiments, optimization engine 152 can determine instabilities by detection of periodic phase synchronizations, which can predict the probability of spontaneous termination without shock. In embodiments, optimization engine 152 can detect phase synchronizations, which can allow optimization of timing of delivery of low energy therapies. Low energy therapies fail if unable there is insufficient myocardium synchronization in chambers of interest. If therapy is timed to a period when significant synchronization is already present, it can have higher probability of achieving defibrillation.

For example, low energy therapy can include anti-tachycardia pacing (ATP) by a series of small electrical impulses delivered to the heart muscle to restore a normal heart rate and rhythm. In another example, low energy therapy can include multistage electrotherapy by a series of low energy biphasic and multiphasic shocks, which is followed by ATP after organization. In another example, low energy therapy can include low energy anti-fibrillation pacing (LEAP). In another example, low energy therapy can include multisite photostimulation.

Input/output engine 150 comprises hardware and software to communicate with electronic devices outside of the system, such as an external server or external programming device (not shown in FIG. 2.) In an embodiment, input/output engine 150 is further configured to communicate with a user of therapy sub-system 100 via, for example, user interface 156. User interface 156 can comprise hardware or software to display the data or signals or evaluations of sensor control engine 150 and/or optimization engine 152.

In one embodiment, sensor 102 and actuator 103 are housed within a single device, in which sensor 102 comprises two or more sensing electrodes, and actuator 103 comprises two or more actuating or effecting electrodes, such as energy delivery electrodes. In a first embodiment, the two or more sensing electrodes are also the two or more effecting electrodes. In a second embodiment, the two or more sensing electrodes are different or separate from the two or more effecting electrodes. In an embodiment, optimization engine 152 is also housed within the same implantable unit with sensor 102 and effector 103.

In an alternative embodiment, at least one of sensor 102, effector 103, and optimization engine 152 are housed within separate units. In one non-limiting example, sensor 102 and optimization engine 152 are housed within a first unit, while effector 103 is housed within a second unit. In an alternative embodiment, sensor 102 and effector 103 are housed within a single or separate implantable device, while optimization engine 152 is housed within an external device, such as a mobile phone, computer, tablet, or other device having a graphical user interface (GUI).

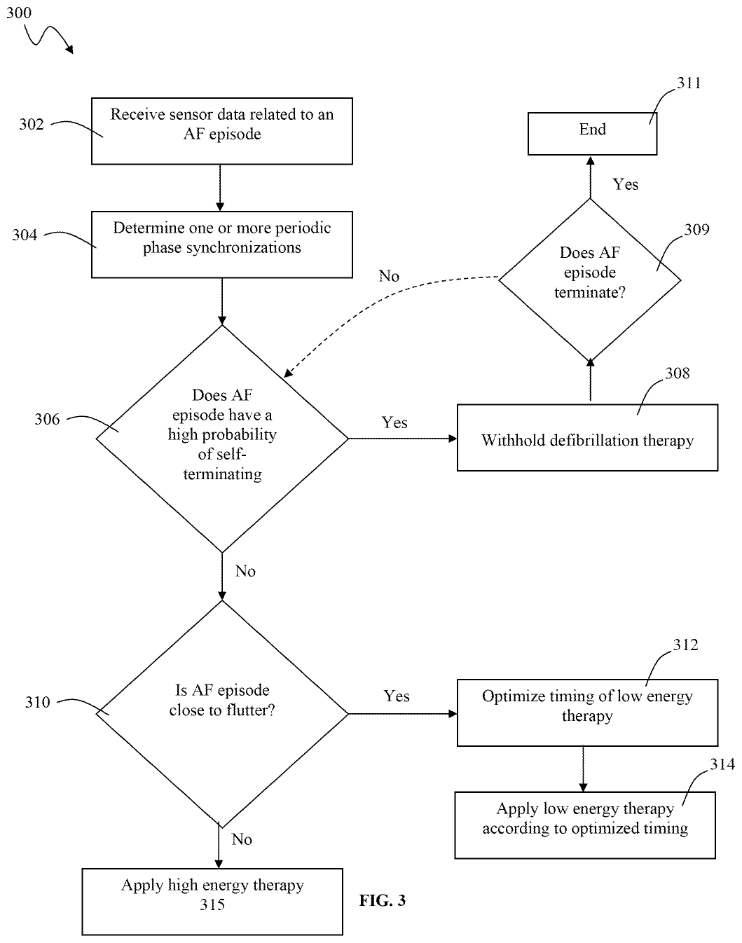

Referring to FIG. 3, a flowchart of a method 300 for optimizing defibrillation related to an atrial fibrillation episode is depicted, according to an embodiment. In embodiments, method 300 can be implemented by therapy sub-system 100 using sensor 102 and effector 103.

Method 300 includes, at 302, receiving sensor data related to an AF episode. For example, sensor 102 can communicate sensor data to sensor control engine 150.

At 304, a determination of one or more periodic phase synchronizations is made. For example, optimization engine 152, using data received via sensor control engine 150, can evaluate the data for periodic phase synchronizations.

At 306, an evaluation of whether the AF episode has a probability of self-terminating based on a predetermined threshold is made. For example, optimization engine 152 can make a probability determination related to self-termination and optionally severity of symptoms of the subject. Probability determinations can be compared against static or dynamic thresholds. For example, if the subject has relatively light or less severe symptoms, a probability of 10% may be suitable in a time period of from anywhere in a range of 1 minute (or less) to 240 minutes or more, and even days may be suitable. If, however, a subject has more severe symptoms, a higher probability of 20% or greater, 30% or greater, 40% or greater, 50% or greater, 60% or greater, 70% or greater, 80% or greater, or 90% or greater may be desired for a time window of 1 minute or less up to 240 minutes or more, depending on the severity of symptoms. In embodiments, the probability threshold and/or time window may be pre-programmed based on a symptom severity rating of the subject (e.g. scale of 1—less severe to 5—most severe).

At 308, if the evaluation at 306 determines a high probability of self-terminating within a period of time, method 300 withholds defibrillation therapy at 308, thereby eliminating even low energy therapy application to patient 10. Method 300 then determines if the fibrillation terminates at 309 within the period of time. If it does, then method 300 ends at 311. If it does not, method 300 returns to 308 to evaluate, based on new data, whether the probability of self-termination remains within the predetermined probability threshold for the period of time.

At 310, if the evaluation at 306 determines that the probability of self-termination is below a threshold, then an evaluation of whether the AF episode is close enough to flutter such that ATP will be highly effective is made.

At 312, if the evaluation at 310 determines that the AF episode is close enough to flutter, an optimized timing of a low energy therapy is calculated and timing is optimized based on extent of organizations. For example, optimization engine 152 can calculate an optimized timing.

At 314, the low energy therapy is applied according to the optimized timing. For example, the low energy therapy can be commanded to sensor 102 using sensor control engine 150.

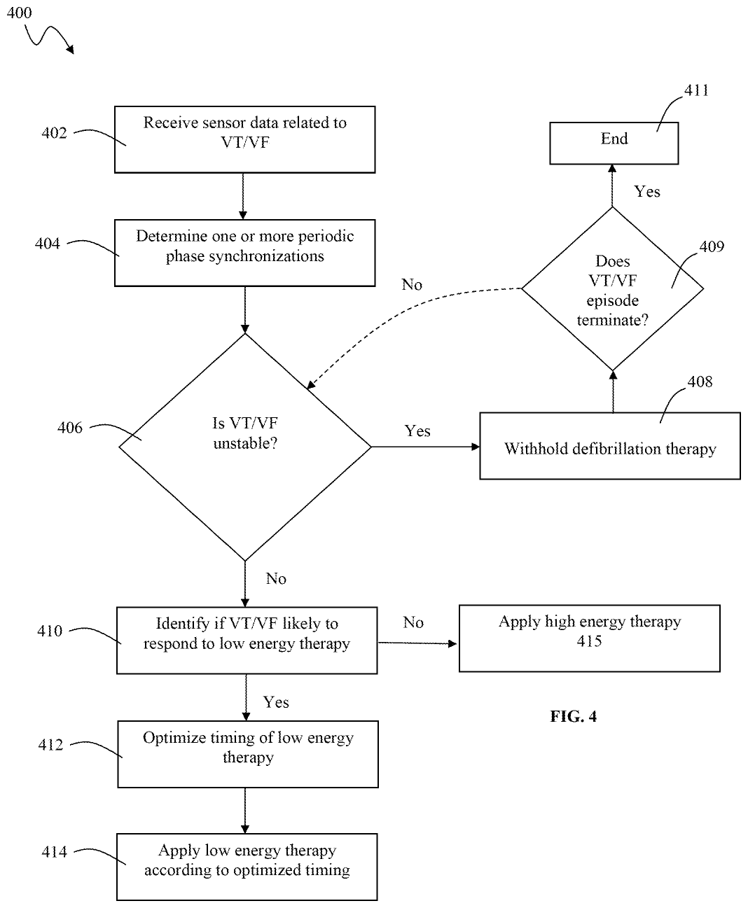

Referring to FIG. 4, a flowchart of a method 400 for optimizing defibrillation related to ventrical tachycardia or ventrical fibrillation is depicted, according to an embodiment. In embodiments, method 400 can be implemented by therapy sub-system 100 using sensor 102.

Method 400 includes, at 402, receiving sensor data related to VT/VF. For example, sensor 102 can communicate sensor data to sensor control engine 150.

At 404, a determination of one or more periodic phase synchronizations is made. For example, optimization engine 152, using data received via sensor control engine 150, can evaluate the data for periodic phase synchronizations.

At 406, an evaluation of whether the VT/VF is unstable is made. For example, optimization engine 152 can make a stability determination. Stability determinations can be compared against static or dynamic thresholds.

At 408, if the evaluation at 406 determines an unstable VT/VF, method 400 withholds defibrillation therapy at 408 for a period of time, thereby eliminating even low energy therapy application to patient 10. Method 400 then determines if the fibrillation terminates at 409 within the period of time. Unlike atrial fibrillation episodes, method 400 must make the determination much quicker, such as a probability of about 80% to about 95% or higher, in a window of time of about 2 to about 10 seconds, or more specifically in a range of about 4 to about 8 seconds. If fibrillation self-terminates within the time period, then method 400 ends at 411. If it does not, method 400 returns to 408 to evaluate, based on new data, whether the probability of self-termination remains within the predetermined probability threshold for the period of time.

At 410, if it is determined that the probability of self-termination within the period of time is at or below the threshold, an identification of whether the VT/VF episode is likely to respond to low energy therapy is made. For example, optimization engine 152 can make an identification of VT/VF likely to respond to low energy therapy.

At 412, an optimized timing of a low energy therapy for a particular VT/VF is calculated. For example, optimization engine 152 can calculate an optimized timing.

At 414, the low energy therapy is applied according to the optimized timing. For example, the low energy therapy can be commanded to sensor 102 using sensor control engine 150.

Referring further to the operation of optimization engine 152, myriad algorithms to determine the probability of self-termination of an AF episode, detect stability of fibrillation, and optimize timing of low energy therapies can be utilized.

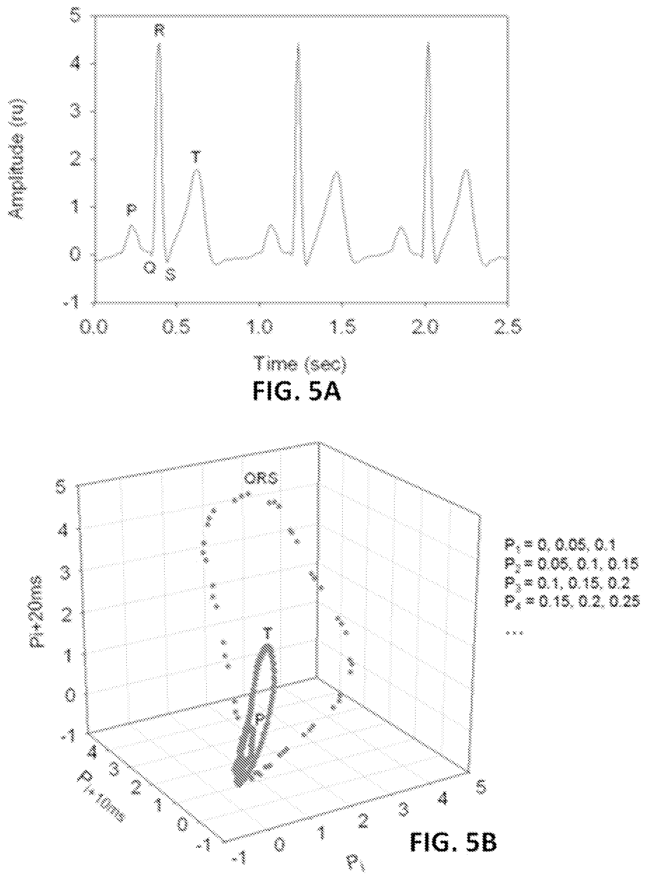

Referring now to FIGS. 5A and 5B, in measuring heart function, a Poincaré plot (FIG. 5B) can be created from an electrocardiogram or ECG (FIG. 5A), embedded in 3D space, i.e. the 1D signal from the ECG is plotted in time series, as shown in FIG. 5B.

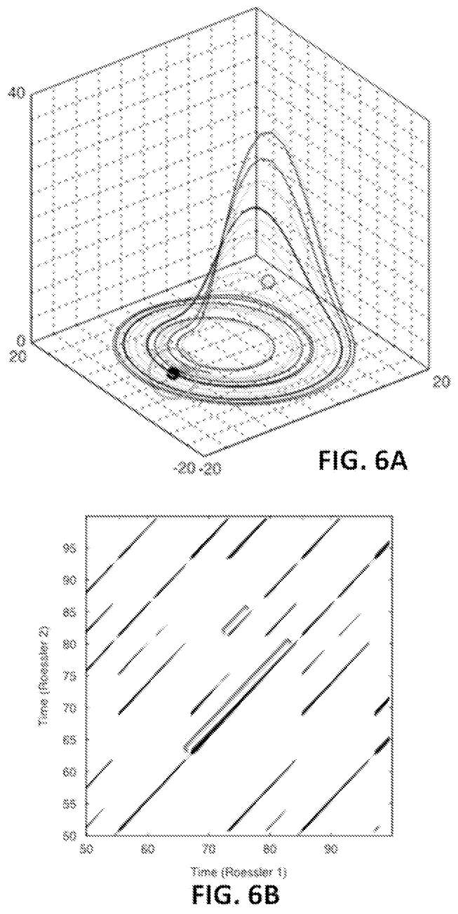

Referring to FIG. 6A, Panel A shows the phase space trajectories of two coupled Roessler systems, one in black and the other in gray. A cross recurrence plot (CRP) of the two systems is displayed in panel B of FIG. 6B. Similar points (e.g. the black and gray circles within a predefined radius of one another) in the two systems will be displayed as black points at the corresponding times in the CRP. Disparate points in the two trajectories (e.g. white circle) are not recurrent and are represented as white in the CRP. Trajectories which remain similar for long periods of time result in long diagonal lines (the longer bracket), whereas trajectories which diverge quickly yield short diagonal lines (the shorter bracket) in the CRP. These figures are adapted from Marwan et al. Physics Reports 2007; 438: 237-329 (hereinafter “Marwan”).

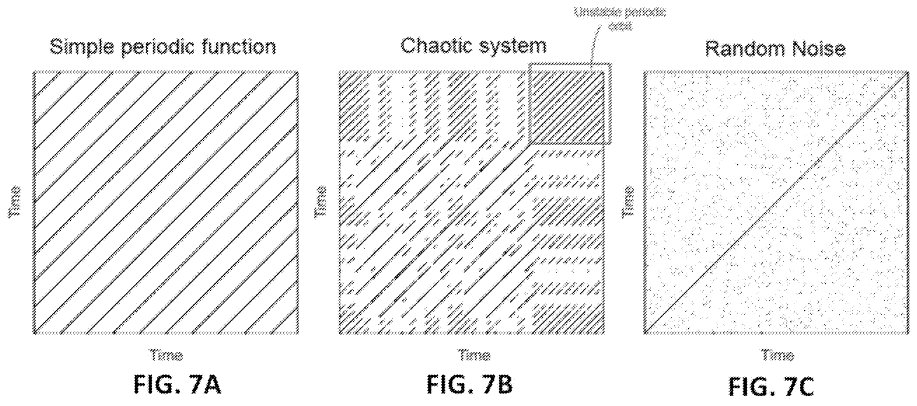

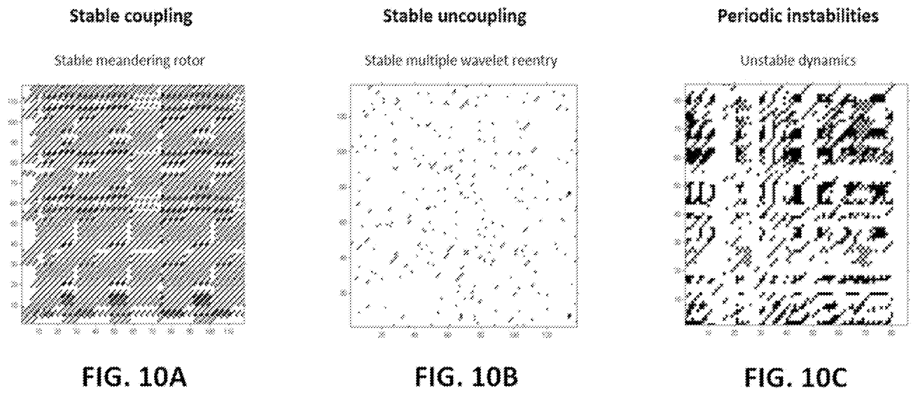

As shown in FIGS. 7A-7C, recurrence plots, also adapted from Marwan, can be interpreted to show a number of trends. For example, for a simple periodic function (predictable), a series of diagonal lines are formed. In a chaotic system, e.g. during fibrillation, there are regions that appear periodic (unstable periodic orbit) which may indicate instability of the wave front such that it may self-terminate or respond well to low energy pacing or shocks if delivered during that time.

Referring to FIG. 8, a flowchart of a method 500 for optimizing treatment related to AF is depicted, according to an embodiment. In embodiments, method 500 can be implemented by therapy sub-system 100 using sensor 102.

At 502, inputs are collected from one or more sensors. In an embodiment, a number of n sensors yielding information about local activation timing in various regions of the atria are collected.

At 504, fiducial points in signals from one or more sensors which identify local activation are identified. In an embodiment, data can be processed to determine local activation timings and intervals between successive activations.

At 506, a determination is made comparing a cycle length to a tachycardia cutoff value. If the determination at 506 is that the cycle length is greater than the tachycardia cutoff value, method 500 returns to 502.

If the determination at 506 is that the cycle length is less than the tachycardia cutoff value, method 500 proceeds to 508. At 508, the extent of coupling is determined. In an embodiment, the extent of coupling is made over one or more rolling windows; for example, rolling windows x seconds long using data from each of the n sensors. The timing intervals, wavefront direction at each site, and the relation of phase from one site to another can be considered. Using such parameters derived from the sensor signals, the extent of coupling present between different regions of the tissue over time can be assessed. Coupling can be measured with tools including phase mapping, cross-recurrence, recurrence networks, synchronization, and coherence. Variables can include, but are not limited to, one or more of determinism, % recurrence, entropy, distribution and trends of diagonal and vertical line lengths, trapping time, Lyapunov exponent, coherence, and phase matching may be calculated over the rolling windows.

At 510, a determination as to tachycardia stability is made. For example, the extent of coupling can demonstrate unstable dynamics (e.g. rapidly oscillating phase synchronizations present between disparate regions) predicting that the abnormal electrical activations may spontaneously terminate without therapy within an acceptable amount of time. In this case, such a determination will trigger method 500 to withhold therapy and wait a reasonable amount of time to assess for spontaneous termination at 512 before applying any shock.

At 514 a determination comparing cycle length and a tachycardia cutoff value is made. If the determination at 514 is that the cycle length is greater than the tachycardia cutoff value, method 500 proceeds to 516 in which the tachycardia is terminated. Accordingly, the tachycardia has terminated without any shock.

If the determination at 514 is that the cycle length is less than the tachycardia cutoff value, an anti-tachycardia therapy is selected at 518. In embodiments, as illustrated, the anti-tachycardia therapy can be selected based on the extent of coupling. For example, macroreentrant tachycardias or reentrant rhythms with one dominant spiral wave and minimal breakup may respond to standard anti-tachycardia pacing. The conditions may be identified by highly stable and synchronized tissue as determined by variables measuring the extent of coupling. The extent of coupling may identify tissue which is amenable to a more benign therapy, ranked in order, for example, antitachycardia pacing <low energy defibrillation <higher energy defibrillation). Accordingly, cutoffs for variables can be assigned, above which a more benign therapy will be selected.

At 520, the selected anti-tachycardia therapy is applied when the extent of coupling during the rolling window is greater than the cutoff for the prescribed therapy.

At 522, a determination comparing cycle length and a tachycardia cutoff value is made. If the determination at 522 is that the cycle length is greater than the tachycardia cutoff value, the tachycardia is terminated at 516.

However, if the determination at 522 is that the cycle length is less than the tachycardia cutoff value, a comparison to a maximum number of attempts threshold value is made. If the maximum number of attempts has not been exceeded, method 500 returns to 508. If the maximum number of attempts is reached at 524, method 500 ends at 526 by stopping therapy.

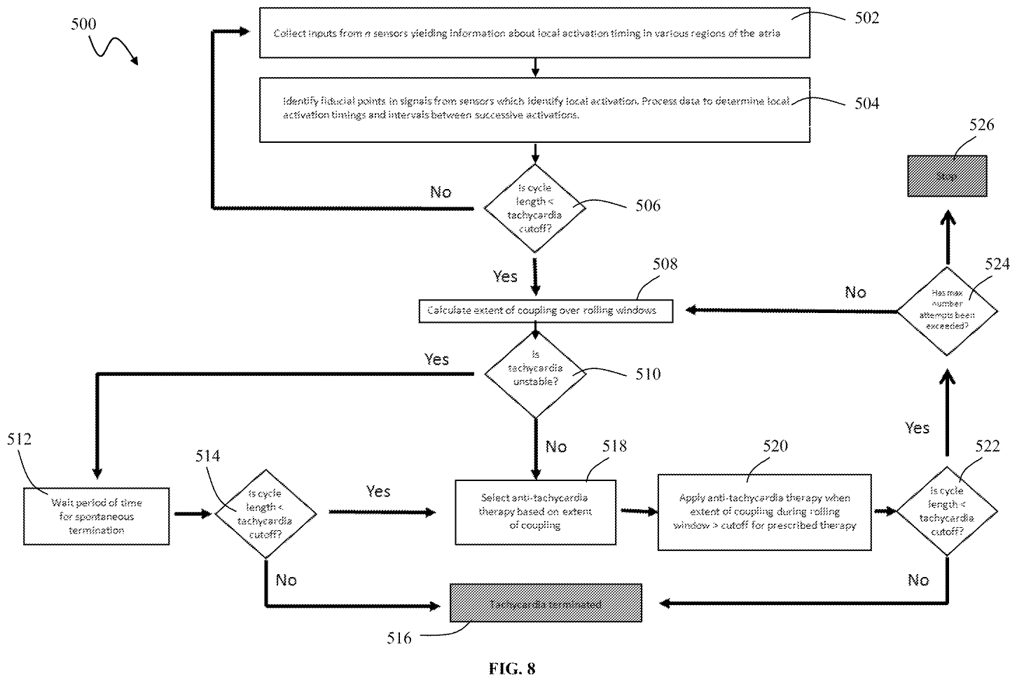

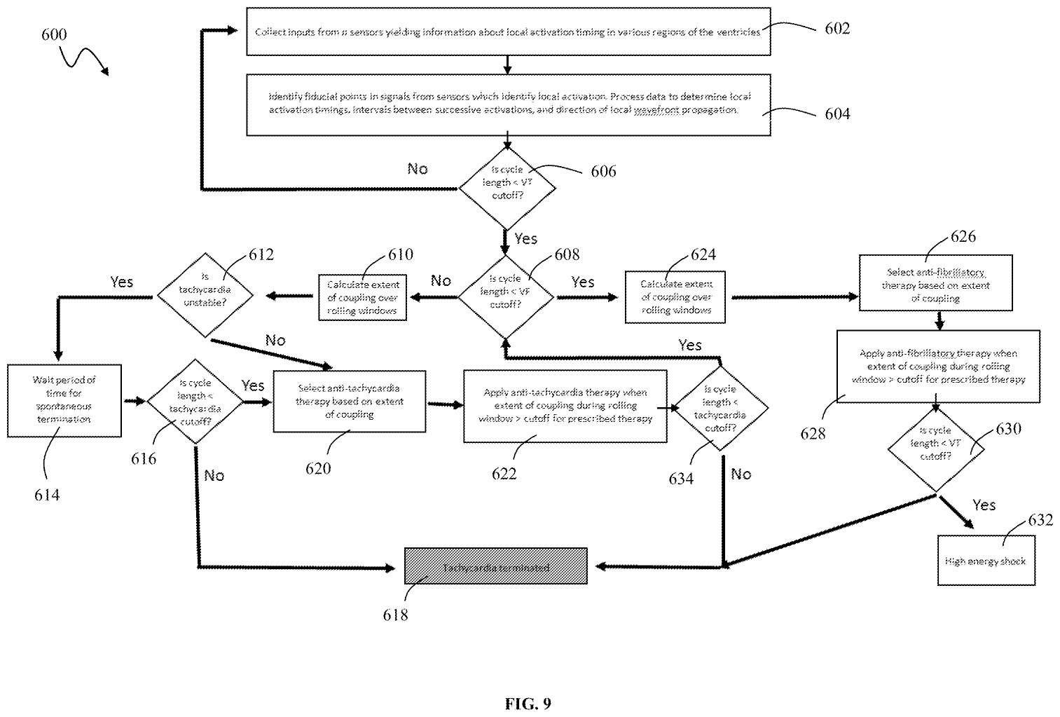

Referring to FIG. 9, a flowchart of a method 600 for optimizing treatment related to VF is depicted, according to an embodiment. In embodiments, method 600 can be implemented by therapy sub-system 100 using sensor 102.

At 602, inputs are collected from one or more sensors. In an embodiment, a number of n sensors yielding information about local activation timing in various regions of the ventricles are collected.

At 604, fiducial points in signals from one or more sensors which identify local activation are identified. In an embodiment, data can be processed to determine local activation timings, intervals between successive activations, and a direction of local wavefront propagation.

At 606, a determination is made comparing a cycle length to a VT cutoff value. If the determination at 606 is that the cycle length is greater than the VT cutoff value, method 600 returns to 602.

If the determination at 606 is that the cycle length is less than the VT cutoff value, method 600 proceeds to 608. At 608, a determination is made comparing cycle length and a VF cutoff value.

If the determination at 608 is that the cycle length is greater than the VF cutoff value, method 600 proceeds to 610. At 610, the extent of coupling is determined. In an embodiment, the extent of coupling is made over one or more rolling windows similar to that described with respect to method 500.

At 612, a determination as to tachycardia stability is made. In an embodiment, stability is determined similar to method 500. If the determination at 612 is that the tachycardia is unstable, method 600 will withhold therapy and wait a reasonable amount of time to assess for spontaneous termination at 614.

At 616, a determination comparing cycle length and a tachycardia cutoff value is made. If the determination at 616 is that the cycle length is greater than the tachycardia cutoff value, method 600 proceeds to 618 in which the tachycardia is terminated. Accordingly, the tachycardia has terminated without any shock.

If the determination at 616 is that the cycle length is less than the tachycardia cutoff value (or, if the tachycardia is stable at 612), an anti-tachycardia therapy is selected based on the extent of coupling at 620.

At 622, the selected anti-tachycardia therapy is applied when the extent of coupling during the rolling window is greater than the cutoff for the prescribed therapy.

At 634, a determination comparing cycle length and a tachycardia cutoff value is again made. If the determination at 634 is that the cycle length is greater than the tachycardia cutoff value, method 600 proceeds to 618 in which the tachycardia is terminated. If the determination at 634 is that the cycle length is less than the tachycardia cutoff value, method 600 returns to 608.

Returning to 608, if the determination at 608 is that the cycle length is less than the VF cutoff value, method 600 proceeds to 624.

At 624, the extent of coupling is determined. In an embodiment, the extent of coupling is made over one or more rolling windows similar to that described with respect to method 500.

At 626, an anti-fibrillatory therapy is selected based on an extent of coupling. Similar to that described above, conditions may be identified by highly stable and synchronized tissue as determined by variables measuring the extent of coupling. The extent of coupling may identify tissue which is amenable to a more benign therapy.

At 628, the timing of therapy is optimized. For example, a selected anti-fibrillatory therapy can be applied when the extent of coupling during a rolling window is greater than a cutoff for a prescribed therapy. In embodiments, cutoffs for variables can be assigned, above which a more benign therapy will be selected. In certain embodiments, variables can be continuously updated that measure the extent of coupling calculated over each rolling window. When the variable exceeds a threshold parameter for a given strategy, this will trigger delivery of therapy.

At 630, a determination is made comparing a cycle length to a VT cutoff value. If the cycle length is less than the VT cutoff value, a high energy shock is applied at 632. However, if the cycle length is greater than the VF cutoff value, method 600 proceeds to 618 in which the tachycardia is determined to be terminated.

Referring now to FIGS. 10A-10C, recurrence plots are interpreted to show stable coupling present in a stable meandering rotor (FIG. 10A) in which the likelihood of spontaneous termination is low, stable uncoupling present in a system in which there is stable multiple wavelet reentry (FIG. 10B), and periodic instabilities present in a system with unstable dynamics (FIG. 10C) in which the likelihood of spontaneous termination is higher.

The invention may be embodied in other specific forms without departing from the essential attributes thereof; therefore, the illustrated embodiments should be considered in all respects as illustrative and not restrictive. The claims provided herein are to ensure adequacy of the present application for establishing foreign priority and for no other purpose.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of U.S.C. § 112(f) are not to be invoked unless the specific terms “means for” or “step for” are recited in a claim.

1. A defibrillation optimizing control system for treating a heart rhythm disorder in a subject, the system comprising:
a sensor operably coupled to the subject and configured to collect data from the subject; and
a therapy control sub-system configured to receive data from the sensor, wherein the data comprises heart activity signals of the subject;
wherein the therapy control sub-system is configured to analyze the heart activity signals to detect an arrhythmia, and when an arrhythmia is present, the therapy control sub-system is configured to detect dynamics or phase synchronizations to determine a probability of the arrhythmia self-terminating within a period of time, and based on the probability of self-terminating, to determine a defibrillation therapy, and
wherein the therapy control sub-system is configured to transmit information to effect a defibrillation therapy within the subject or withhold a defibrillation therapy for a period of time.

2. The system of claim 1, wherein the therapy control sub-system is configured to transmit information to withhold the defibrillation therapy when the probability is above a first threshold.

3. The system of claim 2, wherein the heart rhythm disorder is atrial fibrillation and the first threshold is in range from about 10% to about 90%, and the period of time is in a range of about 10 seconds to 240 minutes.

4. The system of claim 2, wherein the heart rhythm disorder is ventricular fibrillation and the first threshold is about 90% or greater, and the period of time is in a range of about 1 to about 10 seconds.

5. The system of claim 2, wherein, when the probability is at or below the first threshold, the sub-system is configured to determine an extent of organization, and to transmit information to effect delivery of a first therapy or to effect delivery of a second therapy depending on the extent of organization.

6. The system of claim 5, wherein the first therapy comprises energy delivery at a first energy level.

7. The system of claim 6, wherein the second therapy comprises delivery of a second therapy at a second energy level greater than the first energy level.

8. The system of claim 6, wherein when the first therapy is selected, the system is configured to optimize a delivery time of the first therapy at the first energy level.

9. The system of claim 8, wherein the delivery time is at periods of tissue synchronization.

10. The system of claim 5, wherein the first therapy comprises anti-tachycardia pacing by a series of electrical impulses delivered to a heart muscle of the subject to restore a normal heart rate and heart rhythm for the subject.

11. The system of claim 5, wherein the first therapy comprises multistage electrotherapy by a series of biphasic and/or multiphasic shocks, followed by anti-tachycardia pacing by a series of electrical impulses delivered to a heart muscle of the subject to restore a normal heart rate and heart rhythm for the subject.

12. The system of claim 5, wherein the first therapy comprises one of low energy anti-fibrillation pacing and multisite photostimulation.

13. (canceled)

14. The system of claim 1, wherein the heart activity signals comprise intracardiac electrogram signals.

15. The system of claim 1, wherein the sub-system is configured to analyze the signals using real-time recurrence quantitative analysis.

16. The system of claim 1, wherein the signals are analyzed to determine an extent of signal coupling present between different regions of heart tissue over time.

17. The system of claim 16, wherein the extent of signal coupling is measured using one or more tools selected from the group consisting of phase mapping, cross-recurrence, recurrence networks, synchronization, coherence, and combinations thereof.

18. The system of claim 16, wherein the extent of signal coupling is measured using parameters obtained from recurrence networks, multivariate recurrence plots or joint recurrence plots including synchronization measures derived from these plots including correlation of probability of recurrence and joint probability of recurrence.

19. The system of claim 16, wherein the extent of signal coupling is measured using one or more tools selected from the group consisting of synchronization measures including Kuramoto order parameter, interdependence index, network transitivity, and cross transitivity.

20. The system of claim 17, wherein variables measured by the one or more tools are selected from the group consisting of determinism, % recurrence, entropy, distribution and trends of diagonal and vertical line lengths, trapping time, Lyapunov exponent, coherence, phase matching, and combinations thereof.

21. The system of claim 1, wherein the sub-system comprises circuitry within an implantable device, wherein the implantable device is configured to deliver the defibrillation therapy.

22. The system of claim 1, wherein the sub-system comprises an external device wirelessly couplable to the sensor.

23. The system of claim 1, wherein the sensor is configured to collect data from the subject and transmit information to effect the defibrillation therapy within the subject.

24. The system of claim 1, the system further comprising:
an effector separate from the sensor, the effector being configured to effect the defibrillation therapy within the subject.

25. The system of claim 1, wherein at least one of the effector and the sensor comprises at least two electrodes.

26. (canceled)

27. The system of claim 25, wherein the at least two electrodes of the sensor, the effector, or both comprises leads.

28. The system of claim 1, wherein the sensor and the subsystem comprise a single implantable device or two separate devices.

29. (canceled)

30. The system of claim 28, wherein at least one of the two separate devices is implantable within the subject.

31. The system of claim 24, wherein the sensor, the effector, and the subsystem comprise a single implantable device or at least two separate devices.

32. (canceled)

33. The system of claim 31, wherein at least one of the two separate devices is implantable within the subject.

34.-53. (canceled)
